(19) United States
(12) Patent Application Publication
(10) Pub. No.: US 2019/0209078 A1
(43) Pub. Date: Jul. 11, 2019

(54) GROOMING INSTRUMENT CONFIGURED TO MONITOR HAIR LOSS/GROWTH WITH VARIED BRISTLE SPACING

(71) Applicant: L'OREAL, Paris (FR)

(72) Inventors: Gregoire CHARRAUD, Levallois-Perret (FR); Geraldine THIEBAUT, Neuilly-Sur-Seine (FR); Helga MALAPRADE, Vincennes (FR); Guive BALOOCH, Clark, NJ (US); Michael HADDAD, Paris (FR)

(73) Assignee: L'OREAL, Paris (FR)

(21) Appl. No.: 15/863,446

(22) Filed: Jan. 5, 2018

(51) Int. Cl. A61B 5/00; G06T 7/00; G06K 9/66; G06K 9/62; A45D 24/16; A46B 9/02; A46B 9/00; A46B 15/00; A61B 5/053

(52) U.S. Cl. CPC A61B 5/448; G06T 7/0016; G06K 9/66; G06K 9/6267; A45D 24/16; A46B 9/023; A61B 2560/0242; A46B 15/0004; A46B 15/0055; A61B 5/0077; A61B 5/053; A46B 2200/104; A46B 9/005

(57) ABSTRACT

An instrumented comb, including bristles protruding from the handle, the bristles spaced apart from each other such that a first set of two or more bristles are spaced by a first distance, a second set of two or more bristles are spaced by a second distance, and a third set of two or more bristles are spaced by a third distance, wherein the third distance is greater than the second distance and the second distance is greater than the first distance. The comb includes a controller receiving signals from: a contact sensor to detect contact with a scalp, and a magnetic compass to determine location on the scalp. The comb uses the signals to record the detected contact information and determined location information as the comb undergoes a brushing gesture over a scalp, measure hair density based on the contact information and location information, and output the measured hair density.

### FIELD OF THE DISCLOSURE

The present disclosure relates generally to a smart comb that may have, for example, instrumented bristles.

### BACKGROUND

Almost everyone experiences hair loss. An average person may lose about a 100 hairs per day. However, some men and women may lose much more than that, resulting in thinning hair. One sign of thinning hair can include shedding large amounts of hair after combing or brushing. Thinning hair may occur simply by falling out. Some amount of hair may get caught in the comb or brush. Thinning hair can be due to brushing habits or scalp issues. For example, aggressive brushing may lead to larger than normal hair loss. Dieting may lead to lack of nutrients necessary for healthy skin, scalp, and hair, leading to thinning hair. Steps may be taken to promote hair growth and control thinning hair.

However, other than some visible signs of thinning hair, there are no accurate ways for people at home to determine whether treatment for thinning hair is leading to an improvement, or whether the amount of hair loss is normal, or above average. Monitoring treatment for thinning hair generally requires a doctor visit, such as special analysis by a dermatologist.

Also, aside from amount of hair loss, other signs of hair health condition include hair density and dryness. Frequent color or heat treatment can lead to dry hair and breakage. Aggressive brushing or combing too hard while hair is wet can be problematic, as it can lead to hair pulling. Lack of nutrients can lead to hair thinning. Proteins and other nutrients promote stronger hair growth.

However, other than visible signs or touch there are no accurate ways for people at home to judge the health condition of their hair.

The foregoing “Background” description is for the purpose of generally presenting the context of the disclosure. Work of the inventor, to the extent it is described in this background section, as well as aspects of the description which may not otherwise qualify as prior art at the time of filing, are neither expressly or impliedly admitted as prior art against the present invention.

### SUMMARY

In an embodiment, an instrumented comb is provided having a handle, the instrumented comb comprising: a plurality of bristles protruding from the handle, the bristles spaced apart from each other such that a first set of two or more bristles are spaced by a first distance, a second set of two or more bristles are spaced by a second distance, and a third set of two or more bristles are spaced by a third distance, wherein the third distance is greater than the second distance and the second distance is greater than the first distance; a power source; and a controller inside the handle receiving signals from: a contact sensor to detect contact with a scalp, and a magnetic compass to determine location on the scalp, and performing measurements using the signals with one or more processors configured to acquire and record the detected contact information and determined location information as the comb undergoes a brushing gesture over a scalp, measure hair density based on the contact information and location information, and output the measured hair density.

In an embodiment, the first set of bristles is for detecting thin hair, the third set of bristles is for detecting thick hair, and the second set of bristles is for detecting medium thickness hair, wherein a number of hairs that remain on the bristles of each set of bristles after a brushing gesture varies depending on whether the hair on a scalp is thick hair or thin hair, and wherein the hair density is measured based on the number of hair remaining in the comb after the brushing gesture.

In an embodiment, the third set of bristles are thicker than the second set of bristles and the second set of bristles are thicker than the first set of bristles.

In an embodiment, the third set of bristles are longer than the second set of bristles and the second set of bristles are longer than the third set of bristles.

In an embodiment, at least one bristle is a probe for the contact sensor.

In an embodiment, the at least one bristle for the contact sensor is made of stainless steel.

In an embodiment, the instrumented comb further comprises: a conductance sensor to detect conductance of hair, wherein the one or more processors acquire and record the detected conductance, and measure hair moisture content at locations of the scalp based on the conductance information and location information.

In an embodiment, at least one bristle is a probe for the conductance sensor.

In an embodiment, the at least one bristle for the conductance sensor is made of stainless steel.

In an embodiment, the instrumented comb further comprises: an accelerometer to detect motion of the comb, wherein the one or more processors wakes up the comb from an off state when motion is detected and turns off the comb when no motion is detected for a predetermined period.

In an embodiment, the one or more processors adjusts sampling frequency of the contact sensor and magnetic compass with changes in motion detected by the accelerometer.

In an embodiment, the instrumented comb further comprises: a force sensor to sense an amount of force applied by a comb bristle and hair, wherein the one or more processors record in a memory an amount of force applied by a comb bristle together with the location information that the force is applied.

In an embodiment, at least one bristle is a probe for the force sensor.

In an embodiment, the at least one bristle for the force sensor is made of stainless steel.

In an embodiment, the instrumented comb further comprises: a remote processor including a database storing historical hair density measurements; and a communications controller, wherein the comb transmits, via the communications controller, the measured hair density to the remote processor, which stores the measured hair density with date and time when the contact information and the location information were recorded in the database.

In an embodiment, the remote processor includes a display, wherein the display displays a trend in hair density over time based on the historical hair density measurements and the measured hair density stored in the database.

In an embodiment, a system is provided further comprising: a mobile device having a processor executing an app and a display, wherein the app retrieves the historical hair density measurements and the measured hair density stored in the database and displays on the display a trend in hair density over time.

In an embodiment, the instrumented comb further comprises: a temperature sensor to obtain ambient temperature; and a humidity sensor to obtain ambient humidity, wherein the database further includes profile information for a person, wherein the remote processor further includes a machine learning processor, which takes as input the stored historical hair density measurements, the measured hair density, the obtained ambient temperature, the obtained ambient humidity, and profile information, and predicts hair density for a future point in time.

In an embodiment, the instrumented comb further comprises: a camera that captures images of the scalp and hair as the comb strokes over the scalp; a remote processor including a database that stores the captured images of the scalp and hair, and a communications controller, wherein the comb transmits, via the communications controller, the captured images to the remote processor, which stores the captured images with date and time when the images were captured in the database.

In an embodiment, the remote processor further includes a machine learning processor, which takes as input one of the captured images of the scalp and hair and outputs a classification of the hair density.

In an embodiment, a method is provided, implemented by an instrumented comb having a handle, the instrumented comb including a plurality of bristles protruding from the handle, the bristles spaced apart from each other such that a first set of two or more bristles are spaced by a first distance, a second set of two or more bristles are spaced by a second distance, and a third set of two or more bristles are spaced by a third distance, wherein the third distance is greater than the second distance and the second distance is greater than the first distance, a power source; a controller inside the handle, a contact sensor, and a magnetic compass, the method comprising: receiving, by the controller, signals from the contact sensor to detect contact with a scalp, and a magnetic compass to determine location on the scalp; and performing measurements using the signals with one or more processors by acquiring and recording the detected contact information and determined location information as the comb undergoes a brushing gesture over a scalp, measuring hair density based on the contact information and location information, and outputting the measured hair density.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a schematic diagram for a smart comb system according to an exemplary aspect of the disclosure;

FIG. 2 is a block diagram illustrating a controller for a smart comb according to an exemplary aspect of the disclosure;

FIG. 3 is a flowchart for operation of a smart comb system according to an exemplary aspect of the disclosure;

FIG. 4 is a block diagram for a computer system according to an exemplary aspect of the disclosure;

FIG. 5 is a block diagram illustrating a machine learning system for hair and scalp pattern recognition according to an exemplary aspect of the disclosure;

FIG. 6 is a schematic diagram of a processing node for an artificial neural network according to an exemplary aspect of the disclosure;

FIG. 7 is a block diagram illustrating a machine learning system for hair density prediction according to an exemplary aspect of the disclosure;

FIG. 8 is a schematic diagram for another smart comb system according to an exemplary aspect of the disclosure.

### DETAILED DESCRIPTION

Referring now to the drawings, wherein like reference numerals designate identical or corresponding parts throughout several views, the following description relates to a smart comb, a system that uses the smart comb for monitoring hair health over time, and a method. Although the described examples relate to a comb, one would understand that the features disclosed herein apply as well to other hair care tools that come into contact with the hair and scalp such as a hairbrush.

In one or more embodiments, the disclosure relates to a smart comb or hairbrush that can evaluate over time a trend in hair loss based on measurements performed seamlessly while combing or brushing, or by way of a specific gesture different from a regular hair combing or brushing gesture. Hair loss can be evaluated over time based on measurements of hair density over time and/or based on the number of hairs remaining in the teeth of a comb or bristles of a brush after combing or brushing.

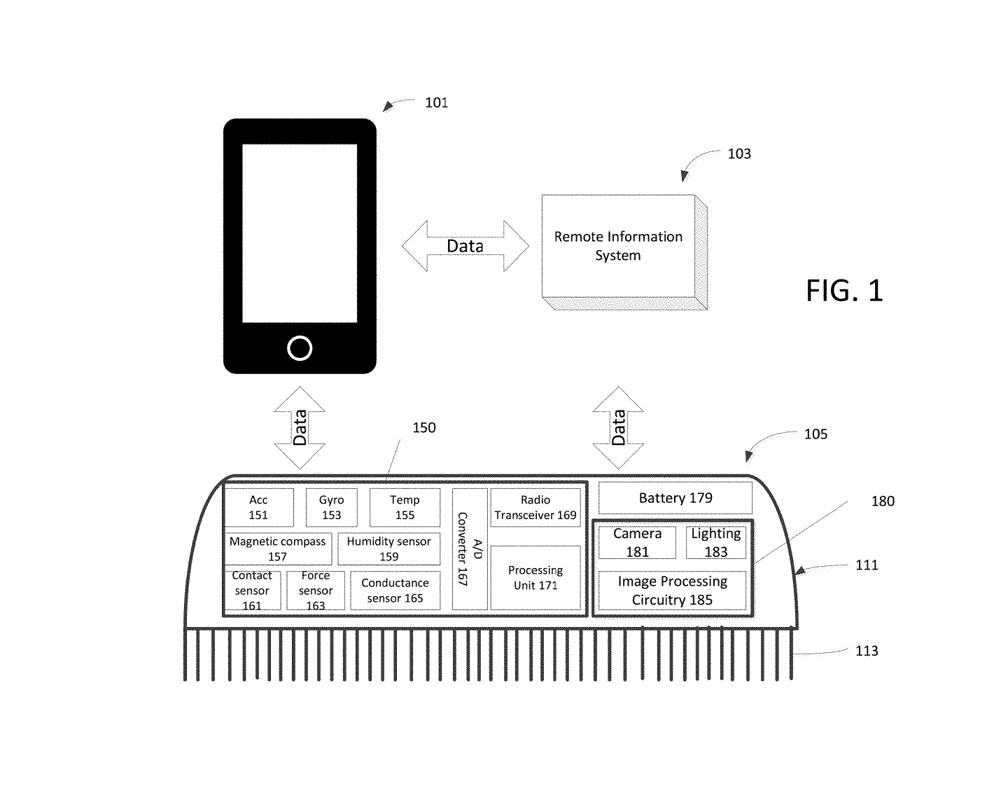

The example smart comb may include several sensors for acquiring data that can be used for making measurements or other calculations to evaluate hair health over time. FIG. 1 is a schematic diagram for a smart comb system according to an exemplary aspect of the disclosure. In some embodiments, the smart comb system may utilize a comb as a sensor that provides some computational capabilities on sensed signals, such as performing conversions and measurements based on sensed signals. In some embodiments, more advanced computations may be performed within the comb, such as performing some image processing functions. In some embodiments, a comb may include power control to save battery life. In some embodiments, a comb may include an optical system for capturing images of the scalp and hair, and in some embodiments may include lighting to support the camera function.

The smart comb system shown in FIG. 1 may include a smart comb 105 that is capable of communicating with external devices so that other processing and display operations may be performed by the other devices. By way of example, the smart comb system may communicate data with a remote information system 103, as well as with a smartphone 101.

Communications may be conducted by any technology that can perform data transfer between devices. In one embodiment, the data transfer may be by way of TCP/IP using WiFi®. Other communication approaches may be used, including, but not limited to, Bluetooth®, or another wireless communication method that can accommodate TCP/IP data transfer. In the case of a hair styling tool that obtains power through a cord, it may be possible to use wired communication.

By way of example, the smart comb 105 in the system shown in FIG. 1 includes a handle 111 and brushing bristles 113. The handle 111 may be a conventional plastic or metal handle, but modified to contain a circuit board 150 having a controller. The circuit board 150 may be completely enclosed inside of the handle, or may be inserted in a slot for easy removal and replacement. The smart comb 105 may be battery powered by a battery 179 so that it takes on the same form as a regular comb without wiring. An embodiment as a hairbrush or other hair care tool may also be battery powered. By incorporating a controller and sensors, the smart comb 105 is capable of collecting data while being used in a conventional combing manner.

In FIG. 1, a contact sensor 161 may detect when the comb is in contact with the scalp and may acquire a hair count as the comb is moved in a regular combing motion. A force sensor 163 may measure the amount of force being applied while the comb is used in a combing motion. A force sensor 163 used in conjunction with a contact sensor 161 may provide information as to whether excessive force is leading to an increase in hair loss.

A conductance sensor 165 may detect if hair is wet or dry. The conductance sensor 165 may perform finer measurements in which the amount of hair moisture may be detected.

Some sensors such as the contact sensor 161, force sensor 163, and conductance sensor 165 utilize particular bristles 113 on the comb to obtained sensory information. For example, the contact sensor 161 includes one or more bristles that when coming into contact with the scalp send one or more signals to a contact sensor circuit. The force sensor 163 may include other bristles 113 that when coming into contact with the scalp may bend. The amount of bending in a bristle for force sensing may be proportional to the amount of force. The conductance sensor 165 may include bristles 113 that detect electrical conductance.

The accelerometer 151 may detect a movement or not movement. Detection of a movement may be used to automatically wake up the smart comb 105. A period without any motion may be used to automatically turn off the smart comb 105. Also, the accelerometer 151 may provide a pattern that is recognized as a specific gesture. The smart comb 105 may use the specific gesture to turn on or off. In an exemplary aspect, the signal from the accelerometer 151 may be used to adjust the sampling frequency of other sensors. For example, as the speed of a combing action increases, the sampling rate may be increased proportionately. Likewise, as speed of a combing action is decreased, the sampling rate may be decreased proportionately.

A magnetic compass 157 may be used to track location of the comb on a scalp. Information on location on the scalp can be used to track measurement of hair density at specific locations so that hair loss measurements are based on measurements at the same location over time.

In an exemplary aspect, the smart comb 105 may include sensors to monitor the environment that the smart comb 105 is being used. Environmental conditions may be measured using a temperature sensor 155 and a humidity sensor 159. In some embodiments, temperature readings and humidity readings may be obtained from an external source proximate to the smart comb 105.

Raw sensor data may be in the form of analog signals. Embodiments of the controller include one or more digital processors 171. An analog-to-digital (A/D) converter 167 is used to convert sensor signals to digital signals that can be handled by the digital processors 171.

In an exemplary aspect, the smart comb 105 includes a communications controller and radio frequency transceiver 169.

Embodiments include an optical system 180 having a camera 181 and optional flash 183 for lighting, and a circuit 185 for handling captured images. The camera 181 may capture images of the scalp and hair as the comb is moved along the scalp. Images may be recorded together with location information obtained from the magnetic compass 157 and date/time information. In some embodiments, photographed images may be periodically transmitted to an external device, such as remote information system 103. In some embodiments, photographed images may be transmitted after completion of image capture.

In FIG. 1, a mobile device 101, such as a smartphone may be in communication with the remote information system 103 and the smart comb 105. The remote information system 103 may be a nearby personal computer, or may be a remote server, or may be a cloud service. The remote information system 103 may include processing that is more powerful than the processing capability available on the smart comb 105. The remote information system 103 may include a database system, which may be used to store data and measurements obtained from the smart comb 105 over time. In some embodiments, the database system stores images transmitted from the smart comb 105 over time.

The mobile device 101 may include an app that can be used to guide a user to obtain data and/or images from the database in the remote information system 103. In some embodiments, the mobile device 101 may display results of analysis performed by the app, or by the remote information system 103. Analysis that may be performed in the remote information system 103 will be described later. In some embodiments, the mobile device 101 may obtain data or images directly from the smart comb 105.

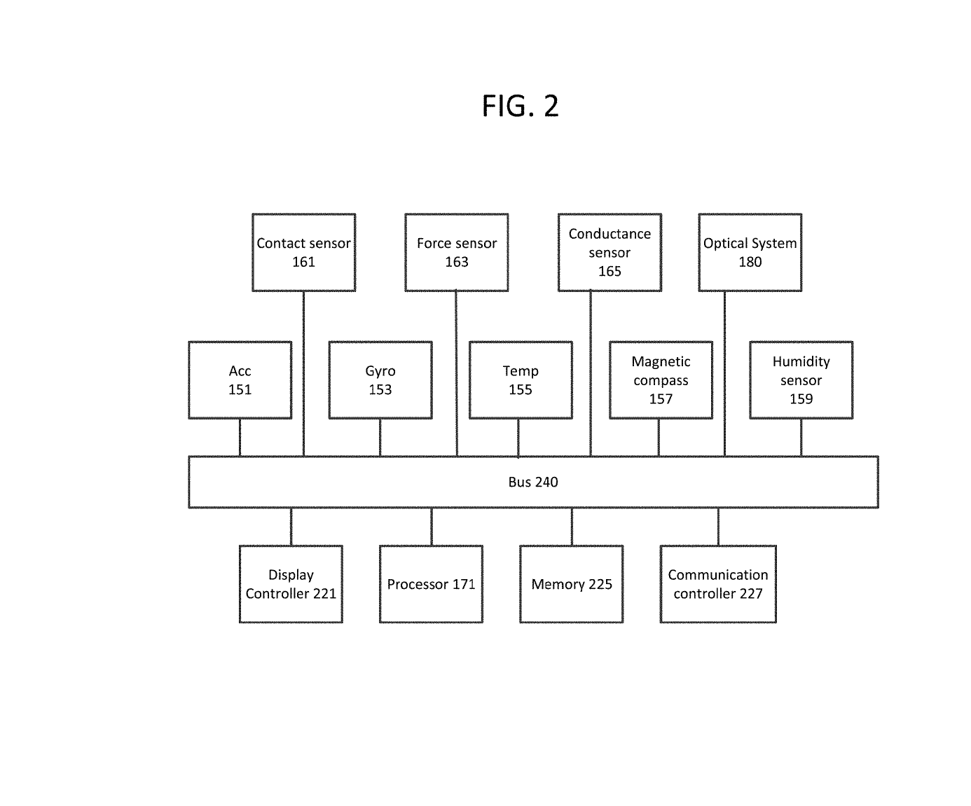

FIG. 2 is a block diagram illustrating a controller for a smart comb according to an exemplary aspect of the disclosure. The controller may be of any available microcontroller having at least a processor core, memory, and programmable input/out peripherals all integrated into a single system-on-chip. The controller may be programmed and the program may be stored in a high-speed memory such as a EEPROM, Flash memory, RAM, or other programmable non-volatile memory. In an exemplary aspect, the controller takes input from any of a number of sensor devices and may include drivers for each type of sensor in order to handle the type of signal associated with the sensor.

The smart comb uses bristles for obtaining the sensory information. Some sensors can take the sensory information from the bristles and convert to signals, which in turn can be converted to digital signals by A/D converter 167 for processing by a processor 171. The contact sensor 161 may be a sensor that detects contact or no contact based on whether a particular bristle or group of bristles come into contact with an object, similar to a push button. In an exemplary aspect, the contact sensor 161 may indicate the amount of contact. An example of a sensor that can detect an amount of contact is a piezoelectric sensor. The force sensor 163 can detect a force on a bristle or a group of bristles. The force sensor 163 may also be a piezoelectric sensor arranged to detect a force. The conductance sensor 165, also referred to as a conductivity sensor, can sense whether the hair can conduct an electric current based on a concentration of ions. The conductance sensor 165 can detect whether the hair is wet or not, or can be configured to detect a relative amount of moisture in the hair. A bristle or a group of bristles may serve as a conductivity probe for the conductance sensor 165.

Other sensors may be implemented internal to the comb handle 111. The accelerometer 151 and gyro 153 may work together to obtain information about motion. The accelerometer 151 is sensitive to forces that cause movement, while the gyro 153 measures orientation. The combination can provide information about the type of motion of the smart comb. The magnetic compass 157 may be based on a magnetometer that can detect magnetic field strength.

In one embodiment, the smart comb may include sensors for detecting environment conditions. The environment sensors may include a temperature sensor 155 and a humidity sensor 159.

In one embodiment, the smart comb may include a digital camera 181 and associated optical circuitry 180. The digital camera 181 may be a semiconductor integrated circuit that converts light into images, such as a charge coupled device (CCD) or pixel sensors.

The circuitry that is associated with the sensors may provide digital signals for storage in a local memory 225 and for processing by a processor 171 via a bus 240. The signals provided by the sensors may be analog signals that are converted to digital signals by A/D converter 167.

In some embodiments, the smart comb may include a communication controller 227 for short range wireless communications. In some embodiments, the smart comb may include some type of visual indication, such as a LED element to show that the power to the smart comb is on. In some embodiments, a display controller 221 may be included so that a small display may display information such as operation status. The small display may be a liquid crystal display (LCD), Light Emitting Diode (LED) display, Organic LED (OLED), or the like, capable of displaying one or more text and/or numeric characters. The small display may be mounted on a flat side of the comb handle 111.

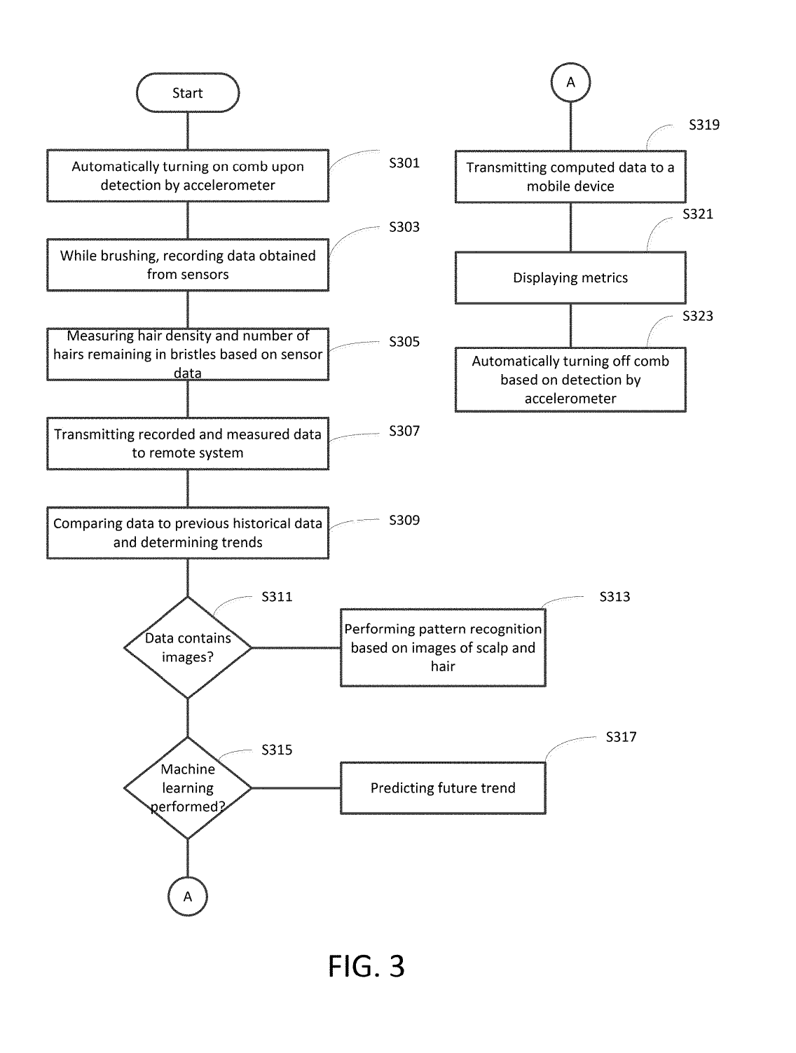

The controller's processor 171 may perform various operations based on one or more of the signals provided by the sensors. Auxiliary information may also be available in the controller such as data/time, some historical information such as a count at a previous time point. FIG. 3 is a flowchart of an exemplary operation of the smart comb 105 in communication with a remote information system 103 and a mobile device having a display as in smartphone 101, as in FIG. 1. In S301, the accelerometer 151 may provide a signal that can be used to determine that the smart comb 105 has been picked up, and as such may automatically move the state from a sleep state to a fully on state. In S303, the contact sensor 161 may send a signal indicating that the smart comb 105 has come in contact with the person's scalp. While in contact, the smart comb 105 may record data obtained from one or more sensors. In an exemplary aspect, the contact sensor 161 may send signals indicating that hair is contacted which can be used to keep an approximate count of the number of hairs. The force sensor 163 may send signals indicating the force that the smart comb is being pressed against the scalp which may provide an indication as to how forceful the smart comb 105 is being used to comb the hair. The magnetic compass 157, together with information from the accelerometer 151 and gyro 153, may provide information of the area of the scalp that is being combed so that location information about the hair count and force by the comb can be included in the recorded information. Similarly, the conductance sensor 165 may sense the amount of moisture in the hair which can be recorded along with the location information to indicate the hair moisture at various locations. Information about the environment at the time of combing can be recorded from the temperature sensor 155 and humidity sensor 159. The recorded data may be used to perform some further measurements. In S305, hair density and number of hairs remaining in bristles may be determined at the completion of a combing session, or periodically when it is determined that an area of the scalp has been combed.

Depending the processing capability and amount of available memory of the controller on the smart comb 105, additional processing may be performed at the smart comb 105. In an exemplary aspect, the recorded data and additional measurements performed on the smart comb 105 are transmitted to a remote information system 103 for additional processing. In some embodiments, the data is transmitted to a mobile device 101 for additional processing and display. For example, the data on the density and amount of hair remaining on the bristles of the smart comb 105 may be transmitted to the mobile device 101 and displayed along with previous density and remaining hair to show a historical trend.

In the case that the data is transmitted to the remote information system 103, in S309, a database system storing historical data for the person may be analyzed against the presently recorded data to determine trends and provide information that indicate how the present data compares to previous historical data. For example, the remote information system 103 may determine whether the hair density is above or below an average of hair density over the historical hair density data. The presently recorded data on number of hairs remaining in the bristles may be analyzed to determine whether the combing action is too harsh or not, or whether excessive hair loss may be partly due to how hard the comb is being pressed during combing.

In some embodiments, the smart comb 105 may include a camera 181 and an optional lighting component 183. The camera 181 may capture images of the hair and scalp during a combing operation. Images may be taken at areas of the scalp, or continuously as combing is occurring. In an exemplary aspect, the camera 181 may be used to take a picture of the overall hair and scalp from a proximate distance from the person's head.

In S311, if images have been, or as images are being captured, in S313, the images may be subject to pattern recognition. The pattern recognition operation may be performed using any of several image processing techniques to determine features contained in the images. In an exemplary aspect, a contrast ratio is determined as a feature of an image of a scalp and hair. The pattern recognition operation may be used to detect hair density at particular areas of the scalp, or over the entire person's head. For example, the contrast ratio may be determined based on a threshold for detection of individual hairs. The image subject to image processing can be used to obtain a count of the hairs in particular areas of the scalp or over the entire person's head.

In one embodiment, machine learning may be applied to obtain machine learning models. The machine learning model may be one that can be trained using temporal data to learn trends, and be used to predict future trends. In S315, if machine learning has been performed, in S317, the machine learning model may be used to predict a future trend, for example, a future trend in thinning hair based on hair density.

In S319, the results of analysis performed by the remote information system 103 may be transmitted to the mobile device 101. The display of the mobile device 101 may be used to display results as metrics to the user.

In S323, a smart comb 105 that includes an accelerometer 151 and a gyro 153 may include a function to automatically turn off when the processor determines that the smart comb has been laid down.

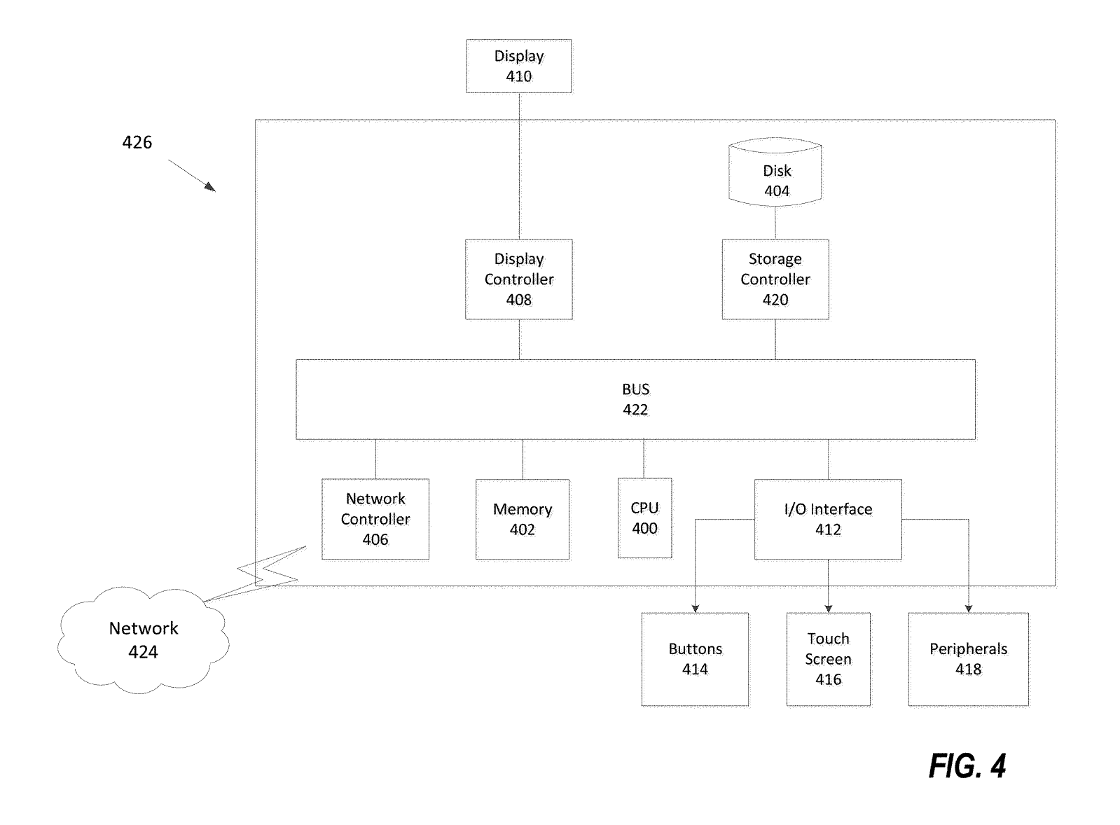

In one implementation, the functions and processes of a computer system for a mobile device 101 or in a remote information system 103 may be implemented by a computer 426. Next, a hardware description of the computer 426 according to exemplary embodiments is described with reference to FIG. 4. In FIG. 4, the computer 426 includes a CPU 400 which performs the processes described herein. The process data and instructions may be stored in memory 402. These processes and instructions may also be stored on a storage medium disk 404 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computer 426 communicates, such as a server or computer.

Further, the claimed advancements may be provided as a utility application, background daemon, or component of an operating system, or combination thereof, executing in conjunction with CPU 400 and an operating system such as Microsoft® Windows®, UNIX®, Oracle® Solaris, LINUX®, Apple macOS® and other systems known to those skilled in the art.

In order to achieve the computer 426, the hardware elements may be realized by various circuitry elements, known to those skilled in the art. For example, CPU 400 may be a Xenon® or Core® processor from Intel Corporation of America or an Opteron® processor from AMD of America, or may be other processor types that would be recognized by one of ordinary skill in the art. Alternatively, the CPU 400 may be implemented on an FPGA, ASIC, PLD or using discrete logic circuits, as one of ordinary skill in the art would recognize. Further, CPU 400 may be implemented as multiple processors cooperatively working in parallel to perform the instructions of the inventive processes described above.

The computer 426 in FIG. 4 also includes a network controller 406, such as an Intel Ethernet PRO network interface card from Intel Corporation of America, for interfacing with network 424. As can be appreciated, the network 424 can be a public network, such as the Internet, or a private network such as LAN or WAN network, or any combination thereof and can also include PSTN or ISDN sub-networks. The network 424 can also be wired, such as an Ethernet network, or can be wireless such as a cellular network including EDGE, 3G and 4G wireless cellular systems. The wireless network can also be WiFi®, Bluetooth®, or any other wireless form of communication that is known.

The computer 426 further includes a display controller 408, such as a NVIDIA® GeForce® GTX or Quadro® graphics adaptor from NVIDIA Corporation of America for interfacing with display 410, such as a Hewlett Packard® HPL2445w LCD monitor. A general purpose I/O interface 412 interfaces with a keyboard and/or mouse 414 as well as an optional touch screen panel 416 on or separate from display 410. General purpose I/O interface also connects to a variety of peripherals 418 including printers and scanners, such as an OfficeJet® or DeskJet® from Hewlett Packard®.

The general purpose storage controller 420 connects the storage medium disk 404 with communication bus 422, which may be an ISA, EISA, VESA, PCI, or similar, for interconnecting all of the components of the computer 426. A description of the general features and functionality of the display 410, keyboard and/or mouse 414, as well as the display controller 408, storage controller 420, network controller 406, and general purpose I/O interface 412 is omitted herein for brevity as these features are known.

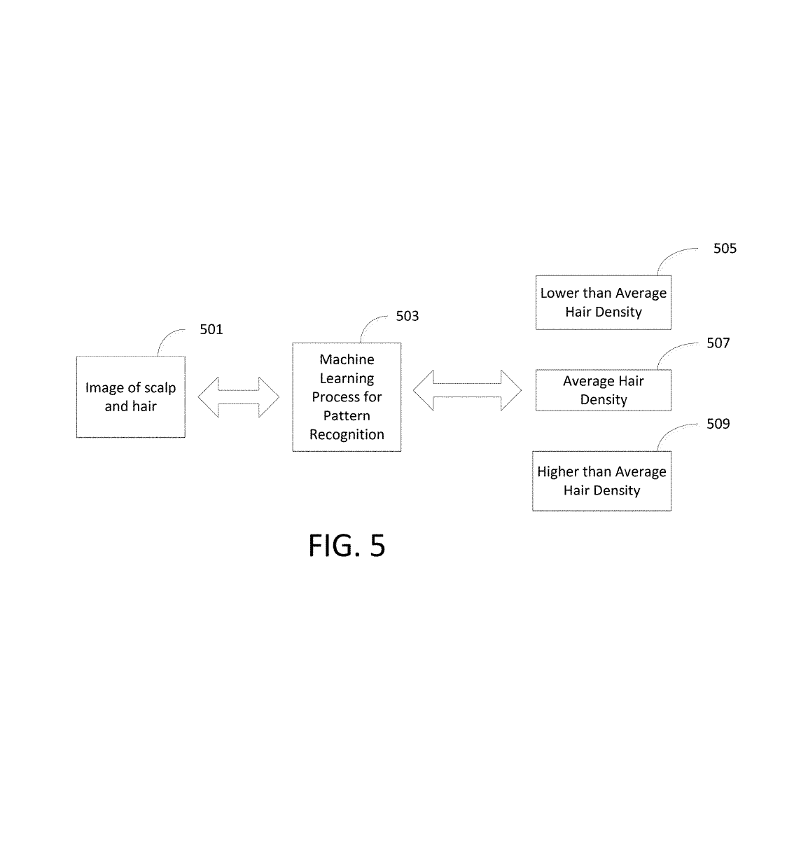

In one embodiment, data and images recorded in a smart comb 105 may be used for machine learning. Machine learning systems can recognize features in photograph images, and the recognition of features can be used to classify an image. In an exemplary embodiment, image processing may be performed to extract features from images of a scalp and hair. The extracted features may be used to train a machine learning system to obtain an accurate hair count. For example, a machine learning system may be trained to distinguish hairs that cross over each other. In an exemplary aspect, a machine learning system may be trained with images of scalps and hair having various hair densities. Each image may be identified as being for a particular hair density category. For example, images of scalps and hair may be identified as being in one of average hair density, thin hair density, or thick hair density. In the one embodiment, in FIG. 5, a machine learning system may be trained for pattern recognition using images of scalps and hair and the trained machine learning system 503 may be provided with an image of a scalp and hair 501 and may determine the category of the image as being average hair density 507, lower than average hair density 505, or higher than average hair density 509.

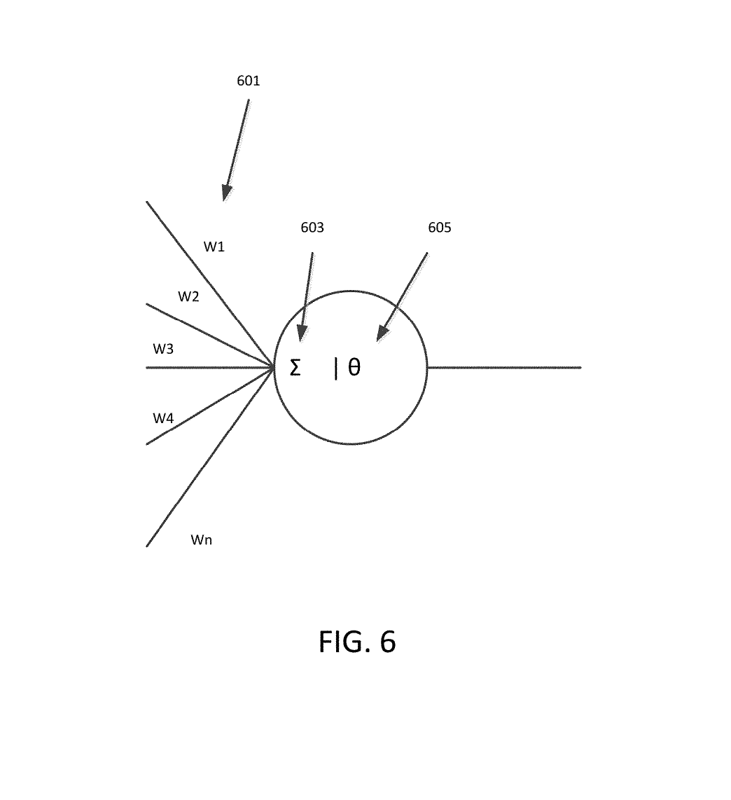

Many types of machine learning systems are capable of being trained to classify images. In an exemplary aspect, preliminary image processing may be performed to identify specific features in a training set of images, and values of the identified features may be used for training a machine learning system. One well known machine learning system is an artificial neural network. A widely used architecture for an artificial neural network is a multi-layered artificial neural network. In some cases, the multi-layered neural network has one or more hidden layers. Each hidden layer may include one or more processing nodes, each of which performs a multiply-add computation. FIG. 6 is a schematic diagram of a processing node for an artificial neural network. Each processing node in a neural network may each perform a weighted sum of inputs from nodes in a previous layer and output a value based on a comparison to a threshold value. In FIG. 6, each input connection is weighted (has an associated weight value W1 to Wn). Each weight value is multiplied by a value associated with the connection, which is an output value of a previous node. The weighted inputs to the node are summed 603, and the sum 603 is compared to a threshold θ 605 in order to determine an output value of the node. During a learning process, the weighted connections 601 between nodes are adjusted in value. When the weighted connections are such that the output error of the neural network is minimal, or within a tolerance level, the neural network is said to be trained to perform a function based on resulting weight values. The trained neural network can perform its trained function for new inputs.

There are several known processes for training neural network architectures, such as the multi-layered neural network mentioned above. One known technique that is capable of training a multi-layered neural network having a hidden layer is the Backpropagation Learning algorithm. See Rumelhart, David E.; Hinton, Geoffrey E.; Williams, Ronald J. (8 Oct. 1986). “Learning representations by back-propagating errors”. Nature. 323 (6088): 533-536, herein incorporated by reference in its entirety. The Backpropagation Learning algorithm is a supervised learning approach, which means that training requires a known output. An objective of training a neural network is to provide a trained system that can generalize. In other words, it is a goal of training to provide a neural network that can perform a function on data that it has not previously been trained. In order to ensure a robust behavior that generalizes for unknown data, it is generally necessary to train a neural network for a large number and variety of training data.

The multiply-add computation that is performed by the nodes of a neural network may be computational intensive. However, specialized graphics processing units (GPU) can perform such a computation efficiently. A GPU included in graphics adapters from NVIDIA, mentioned above, is an example of a processor that may be utilized to perform calculations for a neural network.

In one embodiment, machine learning systems may be trained to predict a future value based on historical data. For example, although a plot of historical values of hair density over time may show a trend, such as that hair density is increasing or decreasing over time, other factors may be taken into consideration in order to predict whether changes in other factors can lead to a different outlook, i.e., a change in the trend. In some cases, the other factors may be taken into consideration is determining whether other factors play a role in the trend. The smart comb 105 of the present disclosure can record information on environmental conditions. A machine learning system may be trained to predict a future value based on a specific environmental condition. A machine learning system may be trained to predict a future value when taking into account a personal feature obtained from a personal profile.

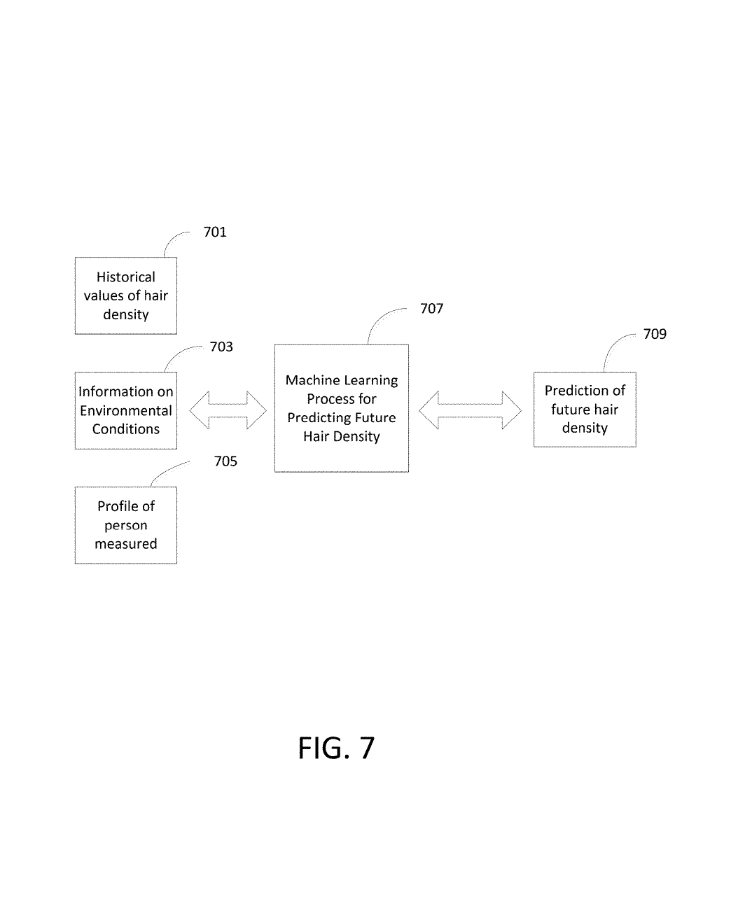

FIG. 7 is a block diagram for a machine learning system for predicting hair density. In an exemplary aspect, a machine learning system may be trained using historical values of hair density 701 together with values of environmental conditions 703 associated with the historical data, and values of characteristics from personal profiles. The values may be obtained from historical data for many persons. The trained machine learning system 707 may be provided with historical hair density values for one person 701 over a number of time periods, information on environmental conditions 703, such as temperature and humidity conditions, under which each of the historical hair density values was obtained, and characteristics from a personal profile 705 for the person whose historical hair density values was obtained. The machine learning system 707 may then predict a future hair density 709. In other embodiments, the machine learning system 707 may predict a future hair density 709 when a present, or alternative, environmental condition is input.

There are several known machine learning approaches for learning from temporal data. However, using supervised learning to predict a trend from historical data requires learning dependence between input and a future value, where the dependence may be non-linear. One approach has been referred to as multi-step prediction, multiple-step or multi-step time series forecasting. See D. M. Kline. Methods for multi-step time series forecasting with neural networks. In G. Peter Zhang, editor, Neural Networks in Business Forecasting, pp. 226-250. Information Science Publishing, 2004.

Machine Learning methods for predicting a trend can be complex and computationally burdensome. As noted above, the machine learning system 707 solves problems associated with multi-step time series forecasting by introducing additional factors including environmental conditions and characteristics obtained from a personal profile in addition to historical data.

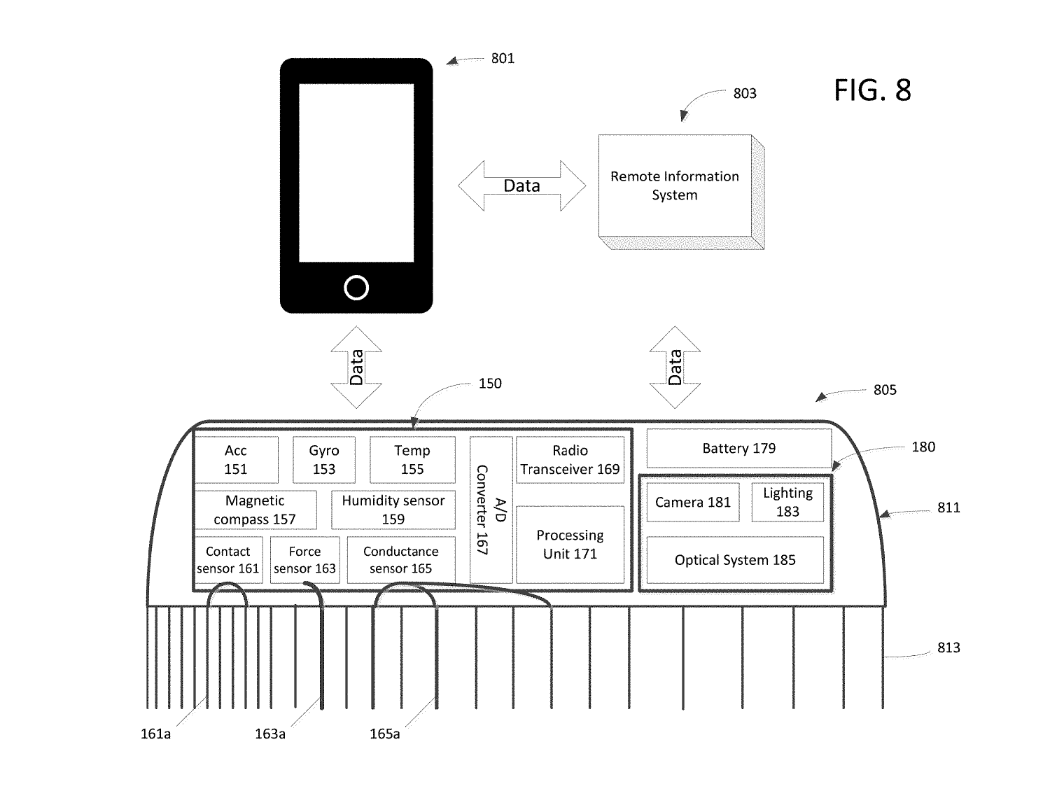

FIG. 8 is a schematic diagram for another smart comb system according to an exemplary aspect of the disclosure. Circuits for various sensors are the same circuits as those in the smart comb of FIG. 1. The smart comb system of FIG. 8 is arranged to obtain data from sensors over a single stroke with minimal error. The smart comb of FIG. 8 has a defined bristle pattern that allows capturing simultaneously several signals at different points of interest spatially, temporally and at a specific movement rate. The defined bristle pattern enables accurate measurement of hair characteristics such as hair density, hair volume, presence of lice, and hair conductance.

Bristles 813 may be of different geometry, size (thickness, length), and material (particular to the type of sensor). For example, the bristles associated with the contact 161 and force 163 sensors may be of different material that the bristles associated with the conductance sensor 165. The bristles associated with the conductance sensor 165 may be of a material having high conductance, whereas the bristles associated with the contact sensor 161 may be of a flexible material. In an exemplary aspect, all bristles associated with sensors are made of a metal, such as a steel alloy. In some embodiments, an increase or decrease in flexibility of bristles may be accomplished by adjustment in the thickness of the bristle.

The defined bristle pattern may obtain an accurate count of the number of hair due to a variable width between adjacent bristles along the comb 805. Bristles 813 of the smart comb 805 detect hair thickness based on spacing between bristles 813. Closely arranged bristles 813 towards the left side of the smart comb 805 detect relatively thin hair, whereas bristles 813 spaced further apart towards the right side of the smart comb 805 detect relatively thicker hair. Bristles 813 in the middle of the smart comb 805 detect hair thickness based on spacing between bristles 813. The number of hairs remaining in the bristles 813 after, for example a single brushing gesture, can be used to measure the hair density. The hair density may be measured in specific areas of the scalp, or over the entire person's scalp.

Also, the accelerometer 151 may detect vibrations during stroking of the smart comb 805, in which case recorded data may include a contact pattern from the contact sensor 161 in combination with a vibration. Several bristles associated with the contact sensor 161 may independently or collectively detect friction that is proportional to a bend in a bristle.

In a case that the smart comb 805 is a brush, a brushing impact may be measured based on a delay between rows of bristles.

A system which includes the features in the foregoing description provides numerous advantages. In particular, hair loss can be measured and evaluated over time at a person's home with a device that preserves the form and usage of a regular comb or brush.

Obviously, numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. An instrumented comb having a handle, the instrumented comb comprising:
a plurality of bristles protruding from the handle, the bristles spaced apart from each other such that a first set of two or more bristles are spaced by a first distance, a second set of two or more bristles are spaced by a second distance, and a third set of two or more bristles are spaced by a third distance, wherein the third distance is greater than the second distance and the second distance is greater than the first distance;
a power source; and
a controller inside the handle receiving signals from:
a contact sensor to detect contact with a scalp, and
a magnetic compass to determine location on the scalp, and
performing measurements using the signals with one or more processors configured to
acquire and record the detected contact information and determined location information as the comb undergoes a brushing gesture over a scalp,
measure hair density based on the contact information and location information,
and output the measured hair density.

2. The instrumented comb of claim 1, wherein the first set of bristles is for detecting thin hair, the third set of bristles is for detecting thick hair, and the second set of bristles is for detecting medium thickness hair,
wherein a number of hairs that remain on the bristles of each set of bristles after a brushing gesture varies depending on whether the hair on a scalp is thick hair or thin hair, and
wherein the hair density is measured based on the number of hair remaining in the comb after the brushing gesture.

3. The instrumented comb of claim 1, wherein the third set of bristles are thicker than the second set of bristles and the second set of bristles are thicker than the first set of bristles.

4. The instrumented comb of claim 3, wherein the third set of bristles are longer than the second set of bristles and the second set of bristles are longer than the third set of bristles.

5. The instrumented comb of claim 1, wherein at least one bristle is a probe for the contact sensor.

6. The instrumented comb of claim 1, wherein the at least one bristle for the contact sensor is made of stainless steel.

7. The instrumented comb of claim 1, further comprising:
a conductance sensor to detect conductance of hair,
wherein the one or more processors
acquire and record the detected conductance, and
measure hair moisture content at locations of the scalp based on the conductance information and location information.

8. The instrumented comb of claim 7, wherein at least one bristle is a probe for the conductance sensor.

9. The instrumented comb of claim 8, wherein the at least one bristle for the conductance sensor is made of stainless steel.

10. The instrumented comb of claim 1, further comprising:
an accelerometer to detect motion of the comb,
wherein the one or more processors wakes up the comb from an off state when motion is detected and turns off the comb when no motion is detected for a predetermined period.

11. The instrumented comb of claim 10, wherein the one or more processors adjusts sampling frequency of the contact sensor and magnetic compass with changes in motion detected by the accelerometer.

12. The instrumented comb of claim 1, further comprising:
a force sensor to sense an amount of force applied by a comb bristle and hair,
wherein the one or more processors record in a memory an amount of force applied by a comb bristle together with the location information that the force is applied.

13. The instrumented comb of claim 12, wherein at least one bristle is a probe for the force sensor.

14. The instrumented comb of claim 13, wherein the at least one bristle for the force sensor is made of stainless steel.

15. The instrumented comb of claim 1, further comprising:
a remote processor including a database storing historical hair density measurements; and
a communications controller,
wherein the comb transmits, via the communications controller, the measured hair density to the remote processor, which stores the measured hair density with date and time when the contact information and the location information were recorded in the database.

16. The instrumented comb of claim 15, wherein the remote processor includes a display,
wherein the display displays a trend in hair density over time based on the historical hair density measurements and the measured hair density stored in the database.

17. A system comprising:
the instrumented comb of claim 15; and
a mobile device having a processor executing an app and a display,
wherein the app retrieves the historical hair density measurements and the measured hair density stored in the database and displays on the display a trend in hair density over time.

18. The instrumented comb of claim 15, further comprising:
a temperature sensor to obtain ambient temperature; and
a humidity sensor to obtain ambient humidity,
wherein the database further includes profile information for a person,
wherein the remote processor further includes a machine learning processor, which takes as input the stored historical hair density measurements, the measured hair density, the obtained ambient temperature, the obtained ambient humidity, and profile information, and predicts hair density for a future point in time.

19. The instrumented comb of claim 1, further comprising:
a camera that captures images of the scalp and hair as the comb strokes over the scalp;
a remote processor including a database that stores the captured images of the scalp and hair;
and
a communications controller,
wherein the comb transmits, via the communications controller, the captured images to the remote processor, which stores the captured images with date and time when the images were captured in the database.

20. The instrumented comb of claim 19, wherein the remote processor further includes a machine learning processor, which takes as input one of the captured images of the scalp and hair and outputs a classification of the hair density.

21. A method, implemented by an instrumented comb having a handle, the instrumented comb including a plurality of bristles protruding from the handle, the bristles spaced apart from each other such that a first set of two or more bristles are spaced by a first distance, a second set of two or more bristles are spaced by a second distance, and a third set of two or more bristles are spaced by a third distance, wherein the third distance is greater than the second distance and the second distance is greater than the first distance, a power source; a controller inside the handle, a contact sensor, and a magnetic compass, the method comprising:
receiving, by the controller, signals from the contact sensor to detect contact with a scalp, and a magnetic compass to determine location on the scalp; and
performing measurements using the signals with one or more processors by
acquiring and recording the detected contact information and determined location information as the comb undergoes a brushing gesture over a scalp,
measuring hair density based on the contact information and location information,
and outputting the measured hair density.
